(19) United States
(12) Patent Application Publication
(10) Pub. No.: US 2026/0218276 A1
(43) Pub. Date: Jul. 30, 2026

(54) A METHOD OF LOWERING BLOOD PRESSURE

(71) Applicant: POLYGENRX, Sydney (AU)

(72) Inventor: Murray CAIRNS, Sydney (AU)

(21) Appl. No.: 19/149,102

(22) PCT Filed: Jan. 18, 2024
(86) PCT No.: PCT/AU2024/050027

§ 371 Date: Jul. 18, 2025

(30) Foreign Application Priority Data
Jan. 20, 2023 (AU) 2023900136

(51) Int. Cl. C12Q 1/6827; C12Q 1/6883; G16B 20/40; G16H 20/10; G16H 50/30

(52) U.S. Cl. CPC C12Q 1/6827; C12Q 1/6883; G16B 20/40; G16H 20/10; G16H 50/30; C12Q 2600/156; C12Q 2600/158; C12Q 2600/172

(57) ABSTRACT

Disclosed herein are methods for the precision treatment of human subjects to lower blood pressure with interventions related to lowering sodium and/or raising potassium, comprising quantifying enrichment of common variation amongst genes involved with sodium and/or potassium biology. As described herein, the quantification of common variant enrichment amongst the genes biologically linked to sodium and/or potassium provides a means of summating an individual's exposure burden to genetics associated with blood pressure that is potentially treatable by an intervention that modulates sodium and/or potassium. In other words, the quantified burden of genetic risk amongst genes linked to sodium and/or potassium may inform individuals more sensitive to treatment by an intervention related to sodium and/or potassium to lower blood pressure.

### FIELD

This disclosure relates generally to methods for lowering blood pressure in a human subject. Specifically, this comprises constructing a blood pressure genetic risk score orientated around genes related to the biology of sodium and potassium, and thus, identifying individuals who may receive increased antihypertensive benefit from lowering their sodium and/or raising their potassium.

### BACKGROUND

Hypertension is a complex disorder characterized by elevated blood pressure.

Genetic risk plays an intrinsic role in blood pressure and hypertension and provides insights that may improve patient outcomes. Although, genome-wide association studies (GWAS) have revealed much of the complexity of the heritable component of blood pressure, we will need innovative approaches to translate vast amounts of genetic data available into clinically actionable insights.

Hypertension affects approximately 1.3 billion adults over 30 years of age worldwide. Risk for hypertension has a strong genetic component, whilst modifiable components of risk pertain to factors like diet, particularly excess intake of sodium and inadequate intake of potassium-rich foods like fruits and vegetables (Bazzano et al., 2013, Current Hypertension Reports, 15:694-702). There is a large body of evidence showing that adjusting an individual's ratio of sodium to potassium intake can influence blood pressure, with specific recommendations that the ideal ratio of sodium to potassium is 1:1 or lower (Ndanuko et al., 2021, Advances in Nutrition, 12:1751-1767).

A key aspect of the genetic component of complex disorders like hypertension or complex traits like blood pressure is that inter-individual heterogeneity is pervasive. In other words, the precise genetic risk factors carried by any given patient will be highly variable, and this can result in similarly variable biology being impacted by the genetic architecture of a disorder. Understanding these differences between individuals is likely crucial to facilitate precision management of these disorders and assist in treatment formulation. Conventional approaches that summate genetic risk burden in an individual, such as a polygenic risk scoring (PRS), also commonly referred to as polygenic scoring (PGS), do so by weighting individual alleles carried genome-wide by their association effect size from a well powered GWAS for the trait or disorder in question. These PRS/PGS approaches have demonstrated significant associations with a diverse range of phenotypes at the population level; for example, heart disease, breast cancer, type 2 diabetes, and inflammatory bowel disease (Khera et al., 2018, Nature Genetics, 50:1219-1224).

This inter-individual heterogeneity is also evident regarding the response of blood pressure to interventions that seek to lower an individual's sodium-to-potassium ratio (Filippini et al., 2021, Circulation, 143:1542-1567). Whilst genome wide PRS/PGS can model individual differences in genetic risk, a key limitation of these methods is their composition of heterogeneous genetic risk factors that lack biological salience and cannot provide specific information that would assist to formulate treatment for a complex disorder like hypertension or to lower blood pressure. As a result, there is an ongoing need for methodology that utilise the genetic architecture of complex disorders like hypertension revealed by GWAS in a manner that is informative for treatment. Specialized genetic risk scores, termed pharmagenic enrichment scores (PES), are specifically oriented around clinically actionable, that is, targetable by drugs or some other kind of intervention, biological pathways or systems (Reay et al., 2020, Scientific Reports, 10(1):879).

### SUMMARY

This disclosure is predicated on the application of biologically directed polygenic scores, that is, a pharmagenic enrichment score, directed to genes associated with the biology of sodium and potassium to inform precision intervention related to sodium and/or potassium to lower blood pressure. A sodium and/or potassium intervention in this context includes, but is not limited to, a pharmacological agent, lifestyle or dietary intervention, or non-prescription supplement; designed to achieve at least one of the following: lower sodium, raised potassium, or a reduction an individual's sodium-to-potassium ratio.

Accordingly, the present disclosure provides a mechanism for lowering blood pressure in a human subject comprising:
a. identifying genes related to sodium and potassium biology from a plurality of data sources, including but not limited to, biological pathways from ontological databases encompassing processes involved in the absorption, transport, action, or excretion of sodium and/or potassium, genes linked to sodium and potassium via evidence amassed in scientific literature, or genes for which expression or function is correlated with treatment by sodium, potassium, or a pharmacological agent that modulates either of these;
b. obtaining data representing genome-wide variant effect sizes from either of the following: a plurality of individuals with hypertension and a plurality of individuals without hypertension, or a plurality of individuals in which blood pressure is measured as a continuous variable, including, but not limited to, systolic blood pressure, diastolic blood pressure, and pulse pressure;
c. selecting a plurality of variants physically mapped to genes, or proximal thereof, from step a, and weighting them by their effect size from the genome-wide variant effect sizes;
d. treating a subject with a sodium and/or potassium intervention, including, but is not limited to, a pharmacological agent, lifestyle or dietary intervention, or non-prescription supplement; designed to achieve at least one of the following: lower sodium, raised potassium, or a reduction an individual's sodium-to-potassium ratio guided by the pharmagenic enrichment score calculated from step c, this comprises;
   i. calculating a pharmagenic enrichment score by summating the variant effect sizes from step c;
   ii. identifying whether the individual's blood pressure will be sensitive to the intervention from step a based on whether the numeric value of the pharmagenic enrichment score is elevated relative to a reference population for which that score is also calculated.

This method of treatment would be used to identify individuals for which an intervention or interventions related to sodium and/or potassium described in step d would exert a greater blood pressure lowering effect.

### BRIEF DESCRIPTION OF THE DRAWINGS

Embodiments of the disclosure are described herein, by way of non-limiting example, with reference to the accompanying drawings.

FIG. 1 displays a non-limiting example of how a pharmagenic enrichment score related to sodium and potassium biology can be constructed for use to lower blood pressure in conjunction with a sodium and/or potassium orientated intervention. The top panel (panel a) is a condensed overview of the methodology applied in this embodiment to implement the pharmagenic enrichment score and evaluate its properties. The next panel displays (panel b) Venn diagrams depicting the overlap in unique genes between different collated ontologies related to sodium and potassium biology. From left to right: sodium transport genes vs potassium transport genes; renal sodium excretion genes vs renal potassium genes; intersection of the four previous gene-sets. The next panel on the left (panel c) denotes tuning genome-wide polygenic scores for SBP and DBP amongst medicated (antihypertensives) and unmedicated participants with measured blood pressure in an independent cohort. The variance explained between the full and covariate only model (ΔR²) or each P value threshold is plotted. The next panel denotes violin plots, with overlaid box-and-whisker plots, of the distribution of measured SBP in each quintile of the SBP PGS in the UKBB cohort. The red dotted line denotes mean SBP in the entire cohort. Panel e denotes Forest plot depicting the effect size (beta estimate with 95% confidence interval error bars) of the tuned PES in the UKBB on SBP and DBP, respectively. The top panel denotes models where genome wide PGS is covaried for, whilst the bottom panel are PGS unadjusted estimates.

FIG. 2 displays the estimated effect size of urinary sodium on SBP at differing values of sodium/potassium transport PES and genome-wide PGS. The estimated effect urinary sodium on SBP in the cohort upon splitting participants into deciles of the Na/K transport PES (panel a) or the genome wide PGS (b), with error bars denoting 95% confidence intervals of the estimate. These blood pressure effect sizes (in mmHg) are per standard deviation (scaled to be one in each decile) for urinary sodium. The dotted line denotes the mean urinary sodium/SBP effect size over all the deciles for either PES or PRS and the grey shaded area indicative of values greater or less than one standard deviation above the mean effect size. A linear trend line is plotted between the per-decile beta estimates to visualize the trend of the effect sizes.

FIG. 3 displays the transcriptional correlates of the sodium/potassium PES and the transport PGS. Specifically, the sodium/potassium transport PES and the genome wide PGS were regressed on the whole blood transcriptome. The correlation between the regression t value (beta/SE) for each gene is plotted transcriptome-wide (a) and specifically within the sodium/potassium transport gene-set (b). A kernel density estimation plot of the distribution of the absolute value of these regression t values is shown transcriptome-wide (a) and specifically within the sodium/potassium transport gene-set (b).

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites, and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

The articles “a”, “an”, and “the” additionally include their plural aspects unless in the event that their context clearly states otherwise. Therefore, reference to “an agent” includes a single agent, as well as two or more agents, and so on, and so forth.

In its central aspect, the present disclosure provides a method for lowering blood pressure whereby it comprises;
a. identifying genes related to sodium and potassium biology from a plurality of data sources, including but not limited to, biological pathways from ontological databases encompassing processes involved in the absorption, transport, action, or excretion of sodium and/or potassium, genes linked to sodium and potassium via evidence amassed in scientific literature, or genes correlated with treatment by sodium, potassium, or a pharmacological agent that modulates either of these;
b. obtaining data representing genome-wide variant effect sizes from either of the following: a plurality of individuals with hypertension and a plurality of individuals without hypertension, or a plurality of individuals in which blood pressure is measured as a continuous variable, including, but not limited to, systolic blood pressure, diastolic blood pressure, and pulse pressure;
c. selecting a plurality of variants physically mapped to genes, or proximal thereof, from step a and weighting them by their effect size from the genome-wide variant effect sizes;
d. treating a subject with a sodium and/or potassium intervention, including, but is not limited to, a pharmacological agent, lifestyle or dietary intervention, or non-prescription supplement; designed to achieve at least one of the following: lower sodium, raised potassium, or a reduction an individual's sodium-to-potassium ratio guided by the pharmagenic enrichment score calculated from step c, this comprises;
   i. calculating a pharmagenic enrichment score by summating the variant effect sizes from step c;
   ii. identifying whether the individual's blood pressure will be sensitive to the intervention from step a based on whether the numeric value of the pharmagenic enrichment score is elevated relative to a reference population for which that score is also calculated.

The proceeding sections will further delineate specific terminology and components of the method for treatment described above.

The term “Complex Disorders” as used herein refers to disorders which do not display typical patterns of Mendelian inheritance in the majority of instances, that is, they do not arise from a single gene or small set of genes. Moreover, complex disorders result from a complex interplay between heritable (genetic) and environmental components. Complex disorders would be known to those skilled in the art, with some examples for illustration including heart disease, schizophrenia, breast cancer, Parkinson's disease, bipolar disorder, diabetes, asthma, and Crohn's disease.

A complex disorder may also encompass one or many “complex traits”, which is often used interchangeably by those skilled in the art with the term “quantitative trait.” These complex traits also do not exhibit Mendelian inheritance patterns, and exist as a distribution of continuous variables amongst individuals-examples thereof include, height, body-mass index, white blood cell count, high-density lipoprotein, blood pressure, and serum creatinine.

Blood pressure would be understood to those skilled in the art to refer to continuous measures, including but not limited to, systolic blood pressure, diastolic blood pressure, and pulse pressure. The state of high blood pressure, as indexed through clinical convention that would be understood to those skilled in the art, is termed hypertension.

The nomenclature Na, Na⁺, K, K⁺ would be understood by those skilled in the art to refer to sodium, sodium ions, potassium, and potassium ions, respectively.

As used herein the terms “treat”, “treating”, “treatment”, “prevent”, “preventing”, “prevention”, “prophylaxis” and the like refer to any and all methods that remedy, prevent, hinder, retard, ameliorate, reduce, delay or reverse the progression of a complex disorder or one or more undesirable symptoms thereof in any way. Thus the terms “treating” and “preventing” and the like are to be considered in their broadest context. For example, treatment does not necessarily imply that a patient is treated until total recovery. Complex disorders are characterized by multiple symptoms, and thus the treatment need not necessarily remedy, prevent, hinder, retard, ameliorate, reduce, delay or reverse all of said symptoms. Methods of the present disclosure may involve “treating” a complex disorder in terms of reducing or ameliorating the occurrence of a highly undesirable event or symptom associated with the complex disorder or an outcome of the progression of the disorder, but may not of itself prevent the initial occurrence of the event, symptom or outcome. Accordingly, treatment includes amelioration of the symptoms of a complex disorder or preventing or otherwise reducing the risk of developing symptoms of a complex disorder.

As understood by those skilled in the art, interventions related to lowering blood pressure may arise in the context of both ‘treatment’ and ‘prophylaxis’—wherein prophylaxis involves strategies to prevent the occurrence of hypertension and/or elevated blood pressure.

The term “variant” as used herein refers to any modification to the DNA sequence as compared to one or more reference DNA sequences. Variants may involve any number of adjacent or spaced apart bases or series of bases, and may include single nucleotide substitutions, insertions, deletions, and block substitutions of nucleotides, structural variants, fusion, copy number variants, repeat length variants, variable number tandem repeats, microsatellites, minisatellites.

In an embodiment, variants are selected from the group consisting of common SNPs, CNV, gene deletions, gene inversions, gene duplications, splice variants and haplotypes associated with the complex disorder. In a preferred embodiment, the variants are SNPs.

The term “genome-wide variants” as used herein refers to information pertaining to genetic variants across the whole genome. Such information includes variants in both coding and non-coding regions of the genome.

In an embodiment, the data representing genome-wide variants is selected from the group consisting of single nucleotide polymorphism (SNP) genotype data, copy number variant (CNV) data, gene deletion data, gene inversion data, gene duplication data, splice variant data, haplotype data, or combinations thereof.

In an embodiment, the data representing genome-wide variants is SNP genotype data.

As used herein, the term “SNP” or “single nucleotide polymorphism” refers to a genetic variation between individuals; e.g., a single nitrogenous base position in the DNA of organisms that is variable. As used herein, “SNPs” is the plural of SNP.

The term “polymorphism” as used herein refers to a locus that is variable; that is, within a population, the nucleotide sequence at a polymorphism has more than one version or allele. One example of a polymorphism is a “single nucleotide polymorphism”, which is a polymorphism at a single nucleotide position in a genome (the nucleotide at the specified position varies between individuals or populations).

The term “gene” as used herein refers to one or more sequence(s) of nucleotides in a genome that together encode one or more expressed molecules, e.g., an RNA, or polypeptide. The gene can include coding sequences that are transcribed into RNA, which may then be translated into a polypeptide sequence, and can include associated structural or regulatory sequences that aid in replication or expression of the gene.

The term “genotype” as used herein refers to the genetic constitution of an individual (or group of individuals) at one or more genetic loci. Genotype is defined by the allele(s) of one or more known loci of the individual, typically, the compilation of alleles inherited from its parents.

The term “haplotype” as used herein refers to the genotype of an individual at a plurality of genetic loci on a single DNA strand. Typically, the genetic loci described by a haplotype are physically and genetically linked, i.e., on the same chromosome strand.

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. One example of a polymorphism is a SNP, which is a polymorphism at a single nucleotide position in a genome (the nucleotide at the specified position varies between individuals or populations).

The term “allele frequency” as used herein refers to the frequency (proportion or percentage) at which an allele is present at a locus within an individual, within a line, or within a population of lines. For example, for an allele “A” diploid individuals of genotype “AA”, “Aa” or “aa” may have allele frequencies of 2, 1, or 0, respectively. One can estimate the allele frequency within a line or population (e.g., cases or controls) by averaging the allele frequencies of a sample of individuals from that line or population. Similarly, one can calculate the allele frequency within a population of lines by averaging the allele frequencies of lines that make up the population.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “locus” as used herein refers to a chromosomal position or region. For example, a polymorphic locus is a position or region where a polymorphic nucleic acid, trait determinant, gene or marker is located. In a further example, a “gene locus” is a specific chromosome location (region) in the genome of a species where a specific gene can be found.

Methods for obtaining data representing genome-wide variants would be known to persons skilled in the art, illustrative examples of which include performing microarray analysis, massively parallel sequencing, amplicon sequencing, multiplexed PCR, molecular inversion probe assay, GoldenGate assay, allele-specific hybridization, DNA-polymerase-assisted genotyping, ligase-assisted genotyping, and comparative genomic hybridization (CGH). Alternatively, data representing genome-wide variants may be obtained from published datasets.

In an embodiment, the data representing genome-wide variants is obtained from genome-wide association study (GWAS) summary statistics.

It is contemplated herein that the data representing genome-wide variants from the plurality of individuals with the complex disorder and the plurality of individuals without the complex disorder may be obtained using one method, which may differ from the method for obtaining data representing genome-wide variants from the subject. For example, SNP genotype data from the plurality of individuals with the complex disorder and the plurality of individuals without the complex disorder may be obtained by SNP microarray, while the SNP genotype from the subject may be obtained by massively parallel sequencing.

In an embodiment, the data representing genome-wide variants from a plurality of individuals with the complex disorder and a plurality of individuals without the complex disorder is obtained from a GWAS. GWAS are observational studies of a genome-wide set of genetic variants in different individuals to see if any variant is associated with a trait. GWAS have identified a large number of genetic variants significantly associated with human disease. These disease-associated variants have provided candidate genes for further study and hypotheses about disease mechanisms. GWAS have also confirmed the polygenic nature of complex disorders, particularly for psychiatric disorders. For example, GWAS studies have demonstrated that the cumulative effect of a large number of weakly associated SNPs, most of which are not statistically significant alone.

The term effect size would be understood by those skilled in the art as an output from a generalized linear model or an analogous statistical approach which represents the effect of a variant, per allele under an additive model, on the phenotype or complex disorder of interest. In an embodiment, these effect sizes represent mean genotype-disorder effects.

A sodium and/or potassium intervention in this context includes, but is not limited to, a pharmacological agent, lifestyle or dietary intervention, or non-prescription supplement; designed to achieve at least one of the following: lower sodium, raised potassium, or a reduction an individual's sodium-to-potassium ratio.

The term pharmagenic enrichment score or PES as used herein refers to a polygenic score calculated for a pharmacologically or clinically relevant set of genes. Specifically annotating total polygenic risk for a disorder in this fashion facilitates a more therapeutically relevant implementation of this information for any given individual. The term “polygenic risk score” is used to define an individuals' risk of developing a complex disorder or progressing to a more advanced stage of a disorder, based on a large number, typically thousands, of common genetic variants each of which might have modest individual effect sizes contribute to the disease or its progression, but in aggregate have significant predicting value. Those skilled in the art would understand that this polygenic risk scoring approach can also be applied in the same fashion for continuous or quantitative traits, but this implementation is usually referred to as a polygenic score, rather than polygenic risk score. In the present case, polygenic risk score may be used to predict the likelihood that an individual will develop a complex disorder using common single nucleotide SNPs associated with the complex disorder, whilst a polygenic score for a continuous trait may be used to predict an individual's measured level of that trait. However, genome-wide polygenic risk scores or polygenic scores (as a biologically unannotated instrument) do not necessarily provide insight into pathways suitable for intervention in individuals.

In accordance with the methods disclosed herein, an elevated PES derived from blood pressure and/or hypertension genetic propensity for gene-sets relevant to sodium and/or potassium biology is indicative that the subject will be sensitive to interventions in terms of reducing blood pressure that lower sodium and/or increase potassium, as described elsewhere herein. As also described elsewhere herein, elevated PES is not significantly related to polygenic risk. Accordingly, the PES approach can capture latent enrichment of polygenic signal in pathways relevant to sodium and/or potassium in subjects with a low overall hypertension PRS and/or blood pressure polygenic score relative to others with the same complex disorder phenotype.

In an embodiment, PES is calculated from SNPs mapped to genes which form the sodium and/or potassium relevant gene-sets, as outlined elsewhere herein. This may comprise model (1) which sums the statistical effect size of each variant in the geneset multiplied by the allele count (dosage) for said variant. For example, for individual i, let [symbol] denote the statistical effect size from the GWAS for each variant j in the geneset, multiplied by the dosage (G) of j in i.

$${PES}_{i} = \sum ( \text{?} \times {G}_{ij} ) \qquad ( 1 )$$

The term “reference predictive polygenic score” is interchangeable with the terms “reference pharmagenic enrichment score” or “reference PES”. In an illustrative example, the comparison may be carried out using a reference predictive polygenic score that is representative of a known or predetermined predictive polygenic risk score from an individual, from a large reference cohort or a cohort of case and controls for the complex disorder phenotype in question, or individuals with a continuous phenotype measured, that is associated with sensitivity to the sodium and/or potassium intervention, as described elsewhere herein.

The reference predictive polygenic score is typically a predetermined predictive polygenic score in a particular cohort or population of subjects. For complex disorders, like hypertension, this may encompass normal healthy controls, subjects with the complex disorder phenotype in question, subjects who had no sign of the complex disorder at the time the reference sample was obtained but who have gone on to develop the complex disorder, etc., whilst for continuous traits like blood pressure any suitable population reference could be used. The reference value may be represented as an absolute number, or as a mean value (e.g., mean+/−standard deviation), such as when the reference value is derived from (i.e., representative of) a population of individuals.

Whilst persons skilled in the art would understand that using a reference predictive polygenic score that is derived from a sample population of individuals is likely to provide a more accurate representation of the predictive polygenic score in that particular population (e.g., for the purposes of the methods disclosed herein), in some embodiments, the reference predictive polygenic score can be a predictive polygenic score derived from the genome-wide variant information obtained from a single biological sample.

The pharmagenic enrichment score or PES in this use, as described elsewhere herein, is calculated specifically relative to genes which are biologically related to sodium and/or potassium biology.

In an embodiment, genes related to sodium and potassium biology may arise from a plurality of data sources, including but not limited to, biological pathways from ontological databases encompassing processes involved in the absorption, transport, action, or excretion of sodium and/or potassium, genes linked to sodium and potassium via evidence amassed in scientific literature, or genes correlated with treatment by sodium, potassium, or a pharmacological agent that modulates either of these.

In an embodiment, PES is calculated from SNPs mapped to genes related to sodium and/or potassium biology, as described above. This may comprise model (1), as defined elsewhere herein, which sums the statistical effect size of each variant in the geneset multiplied by the allele count (dosage) for said variant. In accordance with the methods disclosed herein, an elevated PES for a sodium and/or potassium related gene-set is indicative that the subject will be sensitive in terms of lowering blood pressure to an intervention that includes, but is not limited to, a pharmacological agent, lifestyle or dietary intervention, or non-prescription supplement; designed to achieve at least one of the following: lower sodium, raised potassium, or a reduction an individual's sodium-to-potassium ratio. As described elsewhere herein, elevated PES within sodium and/or potassium relevant genes not significantly related to unannotated polygenic risk across the genome.

The present disclosure will now be further described in greater detail by reference to the following specific embodiment of the construction of PES related to sodium and/or potassium biology to inform identifying individuals who would benefit from an intervention related to sodium and/or potassium, as described elsewhere herein, to lower blood pressure. The following embodiment is provided only be way of example and does not encompass all facets of constructing the PES related to sodium and/or potassium, of which the scope is described elsewhere herein.

### EXAMPLES

### Sodium/Potassium Pharmagenic Enrichment Scores to Direct Interventions Related to Sodium/Potassium to Lower Blood Pressure

Methods relating to this example are henceforth outlined:

In this embodiment, pharmagenic enrichment scores (PES) related to sodium and/or potassium biology are calculated using variant weights from GWAS of systolic blood pressure (SBP) and diastolic blood pressure (DBP), as well genome-wide polygenic scores (PGS) for these two traits. We investigated how these PES and PGS were correlated with blood pressure, and whether an individual's sodium and/or potassium levels, as measured through urinary excretion of these electrolytes—an established biomarker of sodium and potassium intake and levels that would be known to those skilled in the art, modified the relationship of these scores with blood pressure. In other words, if sodium and/or potassium statistically alter the relationship of the PES to blood pressure, this indicates individuals with an elevated PES would be more sensitive to an intervention that lowers sodium and/or potassium, as described elsewhere herein.

Firstly, we describe the methods used to generate the data in this example.

This example is primarily a cross-sectional, observational, study that used genotyped individuals with measured blood pressure available from the UK Biobank (UKBB) cohort. The UKBB is a population-based longitudinal study of approximately 500,000 individuals residing in the United Kingdom aged between 40-69 at recruitment. The primary aim of this study was to explore the interplay between estimated sodium and potassium intake (urinary electrolytes) and genetics on blood pressure, with genetic risk considered genome-wide and as well as pharmagenic enrichment scores (PES) related to sodium/potassium biology. The PES platform aims to partition the polygenic genetic basis for a trait into specific biology related to treatment. In this context, we hypothesized that individuals with elevated genetic load of blood pressure genetic signals amongst specific sodium and potassium pathways may be particularly salient targets to priortise targets for dietary or pharmacological intervention.

The UKBB SNP genotyping procedure and platforms have been outlined extensively elsewhere, with DNA extracted from blood at the baseline visit of participants to a UKBB assessment centre. We processed and performed quality control (QC) on the imputed genotype data resulted in retaining 336,896 unrelated white British ancestry participants, based on kinship estimation and principal components analysis (PCA), respectively. In this subcohort, 13,568,914 autosomal variants survived a series of quality control steps, including, imputation quality filtering (INFO>0.8), minor allele frequency (MAF)>1×10⁻⁴, call rate>0.98, whilst we filtered strong deviations from the Hardy-Weinberg equilibrium.

In the UKBB, we focused on the timepoint where individuals attended one of the UKBB assessment centres for the first time across the United Kingdom for the following: i) study nurse interview and questionnaire completion via touchscreen, ii) a blood draw, iii) a spot urine sample, and iv) anthropometric measurements. We retained participants with non-missing automated systolic and diastolic blood pressure (SBP/DBP) measures (mean of all measures in mmHg) who also had genotyping data available after QC. Individuals who reported taking an antihypertensive at time of assessment (field IDs: 6153, 6177), and thus, blood pressure measurement, were also identified for downstream adjustment.

This cohort was then further filtered to only retain individuals with non-missing urinary biomarker measurements (sodium, potassium and creatinine), with a spot urine measure provided at the same timepoint. Urinary electrolyte analyses leveraged the potentiometric method, whilst creatinine was measured enzymatically, all using a single Beckman Coulter AU5400 clinical chemistry analyzer with the manufacturer's reagents. The urinary sodium and potassium concentrations are reported in the units of millimole per litre, whilst creatinine is available in the units of micromole per litre. A single uniform volume of urine was held and analyzed by the UKBB for all participants (5100 microlitres). Whilst these are spot measurements of urinary sodium and potassium, it has previously been shown that spot samples to perform relatively well in comparison to 24-hour urine collection, particularly in large samples 24,25. Individuals with missing baseline body mass index (BMI) were also excluded. Finally, we excluded a small number of individuals with urinary electrolyte measurements outside the detectable range of the assay, as flagged by the UKBB. This resulted in a full study cohort of 296,476 participants that accorded with all the above. For sensitivity analyses, we tested the variance explained (adjusted coefficient of variation) by each of the following plausible confounders in a separate linear model on raw and natural log transformed SBP, DBP, urinary sodium, and urinary potassium, respectively: age, age², sex, assessment centre attended, month attended assessment centre (seasonality of measurement), 20 single nucleotide polymorphism (SNP) derived principal components (PCs), blood medication as a binary indicator, and BMI. The cross-sectional correlation between urinary electrolytes and blood pressure was estimated using multiple linear regression models (equation 1). Specifically, for participant i let y denote mean SBP or DBP, C′ the transpose of a k×1 vector of covariates, E a scalar denoting the urinary electrolyte, and & the random error term.

$${γ}_{i} ∼ {β}_{0} + {β}_{C} {C}_{i}^{′} + {β}_{E} {E}_{i} + {ε}_{i} . \qquad ( 1 )$$

We tested E as raw values of sodium, potassium, or their ratio (Na:K), as well as models where these electrolyte values were natural log transformed. The primary models were adjusted for age, age², sex, assessment centre attended, month attended assessment centre (seasonality), 20 single nucleotide polymorphism (SNP) derived principal components (PCs), and antihypertensive medication use, whilst we also tested the same models in just unmedicated individuals (N=235,436). We then constructed follow-up models in both the full and unmedicated cohort that also adjusted for BMI. To aggregate the burden of blood pressure associated genetic association at an individual level, we constructed genome-wide polygenic scores (PGS) for SBP and DBP. A genome-wide association study (GWAS) of these two phenotypes was obtained that was independent of the HCS and UKBB to weight the scores from the Genetic Epidemiology Research on Adult Health and Aging (GERA) cohort (Hoffman et al., 2017, Nature Genetics, 49(1):54-56). Specifically, the GWAS was performed on a subset of this cohort of majority European ancestry with relevant blood pressure data available (up to 99,785 participants). Specifically, a PGS in individual i sums the effect size of j variants from the GWAS on blood pressure (\hat{β}ᵢ) multiplied its allelic dosage under an additive model (Gᵢⱼϵ{0, 1,2], equation 2). The full set of M variants included in the score is selected by linkage disequilibrium clumping and thresholding (LD C+T), whereby SNPs are ‘clumped’ such that the retained SNPs are largely independent and ‘thresholded’ based on their association P value in the GWAS.

$${PGS}_{i} = {\sum }_{j = 1}^{M} {\hat{β}}_{j} {G}_{ij} . \qquad ( 2 )$$

The 1000 genomes phase 3 European reference panel was utilized to perform the C+T via a plink v1.9 wrapper implemented by the R ieugwasr package v0.1.528,29. We selected r²=0.1 for clumping in 250 kilobase chunks, with PGS weights at each of the following P value thresholds generated for subsequent tuning (P_{T}): 5×10⁻⁸, 1×10⁻⁵, 1×10-4, 5×10⁻³, 1×10⁻³, 0.01, 0.05, 0.1, 0.5, 1. The summary statistics were cleaned (variants with minor allele frequency <0.01 duplicated variants, strand ambiguous variants (A/T, T/A, G/C, and C/G), and variants tested in <90% of the sample all removed), as well as variants within the extended major histocompatibility complex (MHC) on chromosome 6 due to its LD complexity. We then identified the optimal threshold for score constructing using an external, independent cohort. The null model was the covariates alone (age, sex, and five SNP derived principal components), whilst the full model added the PGS coefficient. We averaged the ΔR² estimated in the medicated and unmedicated cohorts to select the optimal PGS configuration for SBP and DBP to take forward to profile in the UKBB. Plink2 v2.00a3.1 LM 64-bit Intel was utilized to generate the PGS.

Pharmagenic enrichment scores (PES) for SBP and DBP were then constructed specifically in networks/pathways related to sodium and potassium biology. This involved firstly identifying gene-lists related to sodium and potassium biology. We achieved this by searching the molecular signatures database (MSigDB) for ontological pathways related to these factors. Genes from relevant pathways were collated in the following categories nutrient absorption, transport (sodium, potassium, and both sodium and potassium), and renal excretion (sodium, potassium, and both sodium and potassium), totaling seven sets of genes used to construct and tune PES for SBP and DBP. The SBP and DBP summary statistics were filtered for variants overlapping the genes. Thereafter, these variants were then subjected to LD C+T, Tϵ{0.005, 0.05, 0.5, 1}. As outlined in previous work, these thresholds in subsetted scores like PES are designed to capture differing levels of the polygenic signal whilst still retaining enough independent variants such that they are adequately informative of the biology the pathway. After this process, PES are profiled in the same fashion as genome wide PGS but M consists of only clumped variants within the gene-set of interest (equation 4).

$${PES}_{i} = {\sum }_{j = 1}^{M} {\hat{β}}_{j} {G}_{ij} . \qquad ( 4 )$$

Profiling and tuning was identical to the PGS. As a result, we selected the best performing scores from each of absorption, transport, and renal excretion categories to examine in genotyped samples in the UKBB.

The tuned PGS and absorption, transport, and renal excretion PES were profiled in the UKBB cohort described above using PLINK2. We firstly tested the main effects of each PGS and PES on their respective blood pressure measurements using the multiple linear regression approach outlined in equation 1, but with a genetic term instead of the urinary metabolite and the addition of 20 SNP derived principal components (PCs) to control for residual ancestral effects amongst this white British portion of the UKBB. Analyses were performed in both the full and unmedicated cohorts, and scores were scaled to have a mean of zero and unit variance for the purposes of effect size interpretation. To guard against non-specific inflation of the blood pressure genetic signal driving associations between PES and blood pressure, we repeated those PES models additionally adjusted for genome-wide PGS.

We then hypothesized that the joint effect of genetic propensity for blood pressure, either indexed as PGS or a biologically annotated PES, with the urinary electrolytes may significantly depart from additivity, and therefore, constitute a polygenic (gene) by environment interaction (G×E). These analyses were primarily tested in the unmedicated cohort as this subset exhibited more variability in blood pressure, and thus, we are likely better powered to detect non-additive effects. We firstly screened the best performing PGS and PES for SBP and DBP in equation 5, which only includes a G×E term (β_{G×E}GᵢEᵢ).

$${γ}_{i} ∼ {β}_{0} + {β}_{C} {C}_{i}^{′} + {β}_{E} {E}_{i} + {β}_{G} {G}_{i} + {β}_{G \times E} {G}_{i} {E}_{i} + {ε}_{i} . \qquad ( 5 )$$

significant (P<0.05) G×E term with either urinary sodium or potassium were then carried forward for additional sensitivity analyses. As shown previously, spurious G×E effects can be detected when interaction terms between the environmental exposure of interest (urinary sodium or potassium) are not included with all covariates

$$( {β}_{C \times E} {C}_{i}^{′} {E}_{i} ) ,$$

as well as interaction terms between the genetic term and all covariates

$$( {β}_{G \times C} {G}_{i} {C}_{i}^{′} ) .$$

To address this we, we constructed two additional models for G×E pairs with some evidence for non-additivity that controlled for gene-by-covariate (G×C) effects (equation 6), and both G×C and covariate-by-environment (C×E) effects (equation 7).

$${γ}_{i} ∼ {β}_{0} + {β}_{C} {C}_{i}^{′} + {β}_{E} {E}_{i} + {β}_{G} {G}_{i} + {β}_{G \times E} {G}_{i} {E}_{i} + {β}_{G \times C} {G}_{i} {C}_{i}^{′} + {ε}_{i} ; \qquad ( 6 )$$

$${γ}_{i} ∼ {β}_{0} + {β}_{C} {C}_{i}^{′} + {β}_{E} {E}_{i} + {β}_{G} {G}_{i} + {β}_{G \times E} {G}_{i} {E}_{i} + {β}_{G \times C} {G}_{i} {C}_{i}^{′} + {β}_{C \times E} {C}_{i}^{′} {E}_{i} + {ε}_{i} . \qquad ( 7 )$$

For the most significant G×E effect detected in both models we performed additional sensitivity analyses. Firstly, we estimated the effect of the urinary sodium on blood pressure at each decile of either the PES or PGS to screen for evidence that the effect size does not monotonically increase or decrease at differing levels of the environmental exposures. The slopes of the E effect effect amongst participants in the top decile (\hat{β}₁₀) vs each other decile (\hat{β}ₖ) were then sequentially tested for statistically significant differences (equation 8).

$$Z = \frac{( {\hat{β}}_{10} - {\hat{β}}_{k} )}{\sqrt{( {SE}_{10}^{2} + {SE}_{k}^{2} )}} \qquad ( 8 )$$

We also tested the effect of adjusting for urinary creatinine, as well as estimating interactions in the full cohort that covaries for medication status. The statistical significance of G×E effects are also particularly prone to inflation due to heteroskedasticity, and as such, we re-estimated the standard errors as heteroskedasticity consistent (HC) standard errors, specifically leveraging the HC0 (White's Estimator) and HC3 methods via the sandwich R package v3.0-1. Finally, we also considered whether the G term of interest (PGS or PES) was associated with differences in blood pressure variance, rather than just mean effects. Genetic correlates with the variance of quantitative traits have previously been shown to be enriched for factors that display detectable G×E effects (Wang et al., 2019, 5:8). We tested this by splitting the relevant score into deciles, as well as quartiles for a comparison of the leveraged larger sized quantiles, followed by testing for significant differences in variance between these quantiles using Levene's test. A stringent normalization approach was used for this testing of variance effects, in accordance with previous literature related to genetic correlates of variance, whereby the residuals of a model that regressed age, age², 20 principal components, assessment centre, and assessment month on blood pressure were winsorized at five standard deviations above or below the mean, followed by normalization to have a mean of zero and unit variance. This model was constructed in males and females separately to remove mean and variance differences between sexes.

Finally, we investigated the similarity between the gene expression signature (correlation with mRNA expression) associated with the SBP sodium/potassium transport PES and genome wide SBP PGS, respectively. The Genotype-Tissue Expression (GTEx) v8 post-mortem dataset was utilized for this purpose. The transport SBP PES and genome wide SBP PGS were profiled in 838 GTEx individuals sequenced with whole-genome sequencing (WGS) after an extensive quality control (QC) pipeline performed by GTEx, yielding 46,569,704 variants. Of the GTEx tissues available, we focused on whole blood mRNA expression captured by RNA sequencing (RNA-seq) as an exploratory analysis as it is one of the most well-powered tissues in the dataset. Specifically, the effect of these scores was estimated on each transcript drawn from the matrix of normalized expression of 20,247 transcripts amongst 558 individuals with inferred homogenous genetic ancestry that was generated by GTEx for quantitative trait loci estimation (QTL). As outlined previously by GTEx, RNA-seq reads after alignment and initial QC were normalized between samples, lowly expressed genes removed, and expression values subjected to inverse-rank normal transformation across samples. Probabilistic Estimation of Expression Residuals (PEER) normalization was then applied to account for hidden batch effects and other forms of excessive technical or biological variance via estimation of latent covariates (termed PEER factors). In line with the GTEx QTL pipeline, we regressed the PES and PGS separately on each transcript in a linear model covaried for donor sex, five SNP derived PCs, 15 PEER factors, WGS library preparation protocol (PCR-free or PCR-based), and WGS platform (Illumina HiSeq 2000 or Illumina HiSeqX). The correlation between the regression t values (beta/SE) for each transcript with PES or PGS as the explanatory variable, respectively, was tested using linear regression. We estimated this correlation across the entire blood transcriptome and specifically within the genes from the sodium/potassium transport pathway used to construct the PES that were detectable in this dataset. The mean difference in these mRNA signatures between the scores was then tested using a paired t test.

The data pertaining to the above methods are detailed forthwith.

In line with expectation, we demonstrated consistent evidence that urinary electrolytes were significantly correlated with SBP and DBP in the UKBB. In the full cohort, each millimole/L in spot urinary sodium was correlated with a statistically significant increase of 0.025 mmHg [95% CI: 0.024, 0.026] and 0.015 mmHg [95% CI: 0.014, 0.016] in SBP and DBP, respectively. Spot urinary potassium exhibited the converse, with each millimole/L associated with a 0.04 mmHg [95% CI: 0.038, 0.042] and 0.009 mmHg [95% CI: 0.008, 0.01] decrease in SBP and DBP, respectively. We observed that these relationships were consistent considering only participants who did not self-report antihypertensive use at baseline, as well as after BMI was added as a covariate or the electrolytes were natural log transformed. In terms of the ratio of urinary sodium to urinary potassium (Na:K), we saw a monotonic elevation in the estimated positive relationship with blood pressure as the ratio shifted higher above one to be more sodium dominant. These data provide a baseline observational estimate of the correlation between these urinary electrolytes and blood pressure for subsequent genetic stratification.

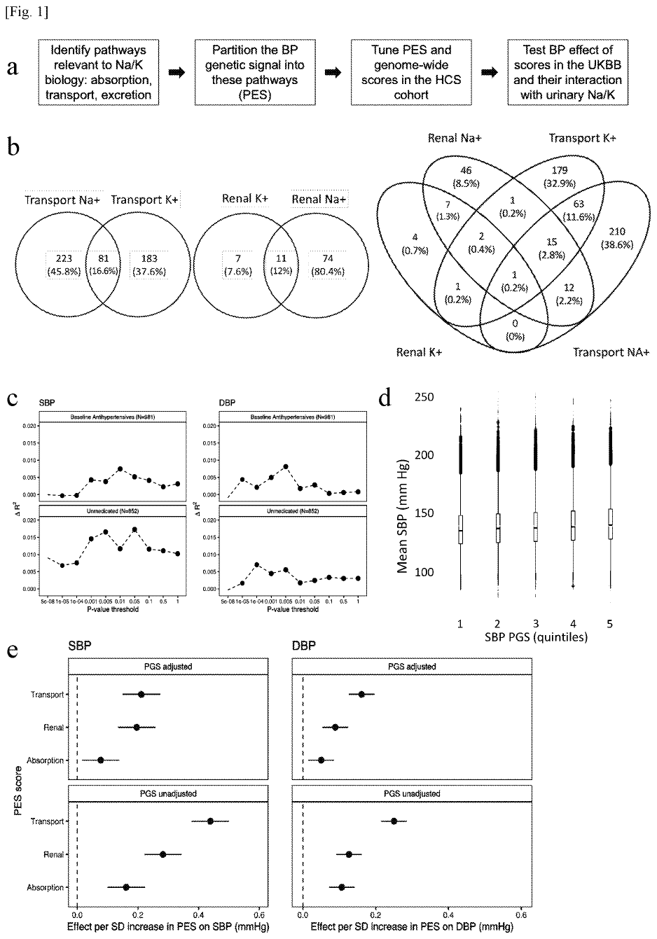

We sought to apply the pharmagenic enrichment score platform to the polygenic signal that influences blood pressure in the context of sodium and potassium biology (FIG. 1a). The PES platform could be theoretically deployed in this context to identify individuals who may receive an outsized benefit from strategies that reduce sodium and boost potassium, including dietary intervention. Pathways from MSigDB were firstly identified and collated to represent broad areas of sodium/potassium biology: nutrient absorption, transport, and renal excretion (FIG. 1b). Genes were aggregated for the nutrient absorption gene-set from four different ontological pathways (64 unique genes), whilst the transport and renal excretion gene-sets were generated for sodium and potassium individually, as well as the genes from pathways relevant to each electrolyte merged. In terms of electrolyte transport, there were 43 sodium transport (304 unique genes) and 37 potassium transport (264 unique genes) pathways, with 487 unique genes once the sodium and potassium gene-sets were merged. Renal excretion processes related to sodium yielded seven pathways (85 unique genes), with four pathways for potassium (19 unique genes), and 93 unique genes once the sodium and potassium excretion gene-sets were merged. This resulted in seven distinct gene-sets used for PES construction: one absorption and three each of transport and renal excretion (sodium alone, potassium alone, and sodium and potassium).

The next stage of the genetic component of the study was to tune each of these PES for the optimum variant configuration with SBP and DBP as the outcome, as well as genome-wide PGS. We utilized the HCS cohort for tuning PGS and PES, which was split into two subsets consisting of participants who self-reported taking an antihypertensive compound and those who did not. Firstly, we profiled genome-wide SBP and DBP PGS at 10 P-value thresholds to identify the threshold for each trait that explained the largest mean phenotypic variance across the medicated and unmedicated cohort (FIG. 1c). For SBP, the largest mean variance explained was at the P<0.01 threshold (mean ΔR²=0.01), whilst a less polygenic score performed optimally for DBP (P_{T}<0.005, mean ΔR²=0.007). As PES from three overarching biology categories were assessed (nutrient absorption, sodium/potassium transport, sodium/potassium renal excretion), we selected the best performing score from each of these three categories for SBP and DBP, respectively. These PES were as follows: SBP-absorption (P_{T}<0.05), sodium and potassium transport (P_{T}<0.005), and renal sodium and potassium excretion (P_{T}<0.5); DBP-absorption (P_{T}<0.005), sodium and potassium transport (P_{T}<1), and renal sodium excretion (P_{T}<1). As a result, we took forward the best PGS and three PES for each of the blood pressure measures at these thresholds and biological annotation.

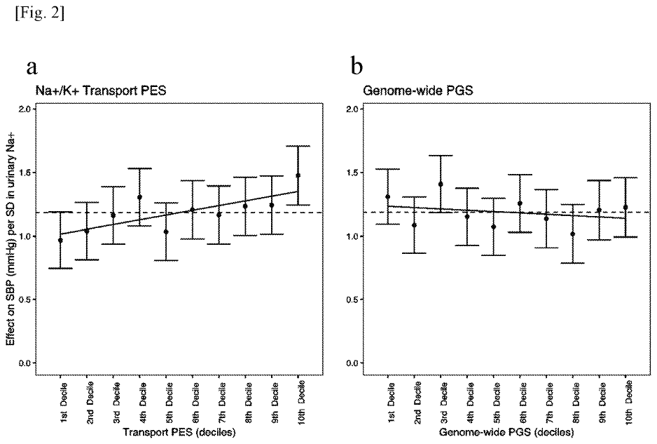

The PGS and PES for SBP and DBP, respectively, were then profiled in the UKBB cohort. Considering the genome wide PGS, we found that genetically proxied SBP and DBP was associated with a larger effect on SBP and DBP than either urinary sodium or potassium alone. For instance, each SD in SBP PGS, was associated with a 1.48 mmHg [95% CI: 1.42, 1.54] increase in SBP, compared to a 1.10 mmHg [95% CI: 1.03, 1.16] increase in SBP per SD of urinary sodium. The effect size of the SBP PGS amongst unmedicated participants had a marginally larger point estimate (1.60 mmHg per SD in PGS) but similar statistical significance as in the full cohort. As visualized in FIG. 2d, each increasing quintile of SBP PGS exhibits higher mean SBP. We see similar results for DBP PGS but at slightly less statistical significance than SBP. We then tested the relationship of the sodium/potassium related PES with measured blood pressure (FIG. 1e). Each of the tuned PES were associated with SBP and DBP correcting for six tests (P<8.33×10⁻³), although the point effect sizes were naturally considerably smaller than the full genome wide PGS, in line with expectation. As a sensitivity analysis, these models were repeated with the additional covariate of genome wide PGS, to control for generalized inflation of the blood pressure genetic signal. The transport and renal excretion PES still had non-zero effects on blood pressure that surpassed multiple-testing correction, whilst the absorption PES became only nominally significant (P<0.05) upon correcting for PGS, suggesting that score exhibits less population-level relevance for blood pressure once the total polygenic burden is accounted for. We emphasize the aim of the PES platform is not necessary to identify overall insights of a phenotype that are generalizable to the population, rather it is designed to be interpreted at an individual level based on their score relative to a population refence. However, PES profiles with statistically significant mean-effects at the population level may have more clinical salience for individuals on the upper tail of the distribution.

We then hypothesized that PES related to sodium/potassium biology are more likely to display a (polygenic) gene-by-environment interaction with measured urinary electrolytes on blood pressure than a biologically undifferentiated genome-wide PGS. This would support the utility of the PES approach to identify individuals who may particularly benefit from dietary or pharmacological interventions to lower sodium and/or raise potassium.

We formally tested this by assessing whether the joint effect of each genetic score (PES or PGS) and the urinary electrolytes on blood pressure significantly departed from additivity in a multiple linear regression model, and thus, constitute evidence for an interaction effect. The screening model for G×E effects contained only one interaction term between the genetic score and the urinary electrolyte tested (Equation 5). The genetic and urinary terms were both scaled to have a mean of zero and unit variance to aid the interpretability of the interaction terms. For genome wide PGS, there was nominal evidence of a non-additive joint effect of SBP PGS and urinary potassium on measured SBP: β_{G×E}=−0.08, SE=0.035, P=0.02. The negative sign of this interaction term suggests that the estimated hypertensive effect of each SD in scaled SBP PGS on measured SBP exhibits a small decrease per unit (SD=1) increase in urinary potassium. This may indicate that boosting potassium intake could lessen the effect of underlying genetic propensity for higher SBP-however, this result must be interpreted cautiously. Firstly, the statistical significance is nominal and does not survive correction for the number of tests performed, and perhaps more importantly, the interaction term becomes non-significant once G×C (PGS-by-covariate, Equation 6) terms are added in the next modelling stage to control for PGS-by-covariate effects (P=0.08). SBP or DBP PGS did not exhibit any other statistically non-zero interactions with either urinary sodium, urinary potassium, or their ratio. The six PES were then screened in the primary G×E model, revealing two nominally significant interaction terms for the DBP renal sodium excretion PES with urinary potassium on DBP and the SBP sodium/potassium transport PES with urinary sodium on SBP. Crucially, both interactions remained significant in the G×C models, as well as the final model that also added electrolyte-by-covariate interaction terms (E×C, Equation 7). Specifically, the estimated effect of urinary sodium on measured SBP increased per SD increase in the SBP sodium/potassium transport PES (β_{G×E}=0.1, SE=0.037, P=8.8×10⁻³), supporting the hypothesis that individuals with higher PES related to sodium/potassium transport may be particularly susceptible to the hypertensive consequences of sodium. The interaction between the renal sodium transport DBP PES and urinary potassium was also positive (β_{G×E}=0.04, SE=0.02, P=0.03). This result may represent a small ablation of the antihypertensive effects of potassium for those with higher PES in this urinary excretion pathway.

As the sodium/potassium transport PES-by-urinary sodium interaction was most significant, we performed a series of additional sensitivity analyses described forthwith. Firstly, we compared the PES-by-sodium interaction with the PGS-by-interaction term directly from the full model outlined in equation 7 and found no evidence that PGS displays an interaction with sodium once G×C and E×C effects are accounted for. This PES also displayed a non-zero positive interaction with scaled sodium: potassium ratio, but not with urinary potassium. We then investigated the effect size of urinary sodium on SBP per decile of the PES versus its effect per decile of the PGS, to support the inferred PES-by-sodium interaction (FIG. 2). As visualised in FIG. 2a, there is evidence of an inflection point at higher deciles the Na⁺/K⁺ transport PES of in terms of magnitude of the positive correlation between the urinary sodium and SBP-in other words, the effect of the sodium on SBP is more pronounced at for participants with higher genetic load in this Na⁺/K⁺ transport pathway, supporting the existence of a G×E effect. We formally compared the urinary sodium effect sizes on SBP in each decile of the PES individually relative to the highest decile and found the hypertensive effect of sodium in the highest decile was statistically significantly larger (P<0.05) relative to the first, second and fifth decile, with a trend (P<0.1) for the third and seventh decile (equation 8, Supplementary Table 9). For instance, each SD in urinary sodium was associated with a 1.48 mmHg increase in SBP the top 10% of the distribution of the PES versus 0.97 mmHg SBP increase in the lowest 10% of the PES. In comparison, the estimated effect of PGS in each of the deciles of urinary sodium was much more consistent, with not even a trend (P<0.1) towards the effect size of urinary sodium in the highest PES decile being significantly different than any of the others.

We also re-estimated the standard errors of this interaction term using Heteroskedasticity consistent (HC) methods, which did not ablate the statistical significance of the departure from additivity. Moreover, the interaction remained significant upon adding terms to adjust for BMI and urinary creatinine, and there was no evidence for PES-by-BMI or PES-by-creatinine effects. There was also evidence that the variance of SBP was significantly different between quantiles of the PES, thus adding support to the existence of non-additive effects given variance related genetic effects on quantitative traits are enriched for detectable G×E. This phenomenon of unequal SBP variance was more pronounced between quartiles (P=5.67×10⁻³, Levene's test), than deciles (P=0.049, Levene's test) of the transport PES. Interestingly, the PES-by-sodium interaction was only non-zero at the optimum threshold after tuning in the HCS (P_{T}<0.005), with no statistically significant effects upon testing transport PES constructed at the remaining thresholds considered (P_{T}<1, P_{T}<0.5, and P_{T}<0.05), which may indicate the effect is only observable at a less polygenic level of the SBP genetic signal or because this PES has a larger mean effect on measured SBP. In the full cohort, where individuals taking antihypertensives were included and adjusted for by an additional covariate term, the transport PES-by-sodium interaction was directionally consistent but not statistically significant, which may be a product of the power loss due to the variance in SBP being reduced upon adding medicated individuals.

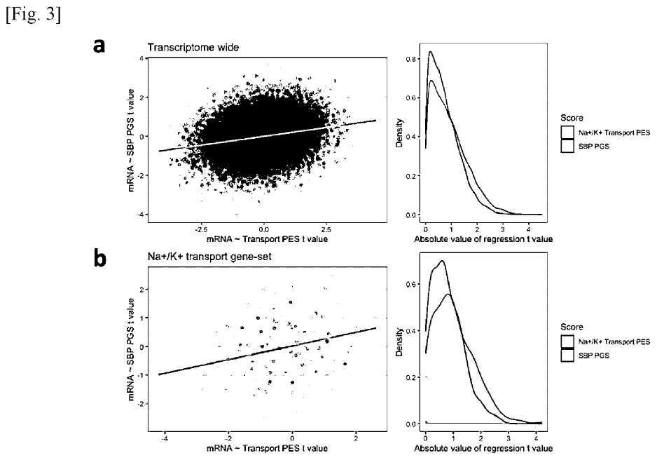

Finally, we compared the transcriptomic signature of the genome wide SBP PGS to the sodium/potassium transport PGS by correlating both these scores with the normalized expression of 20,247 transcripts in whole blood (FIG. 3). Neither PES nor PGS exerted a strong effect in terms of statistical significance (FDR<0.05) on any single transcript, although we are probably underpowered to detect such effects on a per gene basis due to the biological complexity of these scores. We found that the transcriptomic signature of PES and PGS in whole blood was positively correlated with moderate strength across the entire blood transcriptome (r=0.22 [95% CI: 0.21, 0.23]) and specifically within the sodium/potassium transport pathway genes detectable after QC in this tissue (r=0.30 [95% CI: 0.19, 0.40]). However, the PES had a larger mean absolute effects on both mRNA expression transcriptome-wide (((20,246)=27.05, P=2.86×10⁻¹⁵⁸) and localized within the PES (t(286)=4.56, P=7.72×10⁻⁶) gene-set than PGS. This may be because as a more biologically orientated score, the PES has a more direct relationship with the gene expression, either by more readily capturing influences of expression quantitative trait loci acting in cis, or a polygenic effect on expression that exert their impact in trans.

The above embodiments in this example represent evidence that PES constructed using genes related to sodium and/or potassium biology could identify individuals who would be more sensitive to interventions related to sodium and/or potassium, as described elsewhere herein, in terms of lowering blood pressure.

1. A method for lowering blood pressure in a human subject comprising:
a. identifying genes related to sodium and potassium biology from a plurality of data sources, including but not limited to, biological pathways from ontological databases encompassing processes involved in the absorption, transport, action, or excretion of sodium and/or potassium, genes linked to sodium and potassium via evidence amassed in scientific literature, or genes for which expression or function is correlated with treatment by sodium, potassium, or a pharmacological agent that modulates either of these;
b. identifying individuals who may be more sensitive to a sodium and/or potassium intervention, including, but is not limited to, a pharmacological agent, lifestyle or dietary intervention, or non-prescription supplement; designed to achieve at least one of the following: lower sodium, raised potassium, or a reduction an individual's sodium-to-potassium ratio, through the following;
i. obtaining data representing genome-wide variants from a plurality of individuals with hypertension and a plurality of individuals without hypertension, or a plurality of individuals in which blood pressure is measured as a continuous variable, including, but not limited to, systolic blood pressure, diastolic blood pressure, and pulse pressure;
ii. obtaining estimated effect sizes on the disorder from said plurality of variants;
iii. generating a plurality of annotations corresponding to the genes identified in either step (a);
iv. identifying genome-wide variants that intersect the annotations from step (b) (iii);
v. obtaining data representing genome-wide variants in a biological sample from a subject;
vi. calculating a predictive polygenic score for only variants annotated to the sodium and/or potassium gene set identified in step (a) from the subject's genome wide variant data;
vii. identifying individuals more sensitive to the intervention described in step (a) from the predictive polygenic score from step (c) (vi) that is elevated relative to a reference value indicates that the subject may be sensitive to the at least one of the sodium and/or potassium interventions described in step (a);
c. treating the subject with the selected intervention.

2. The method of claim 1, wherein the data representing genome-wide variants is selected from the group consisting of single nucleotide polymorphism (SNP) genotype data, copy number variant (CNV) data, gene deletion data, gene inversion data, gene duplication data, gene fusion data, variable number tandem repeat data, microsatellite repeat data, trinucleotide repeat expansion data, splice variant data, haplotype data, or combinations thereof.

3. The method of claim 1, wherein the data representing genome-wide variants is genome wide association study (GWAS) summary statistics.

4. The method of claim 1, wherein the variants are selected from the group consisting of common SNPs, CNV, translocations, gene deletions, gene inversions, gene duplications, gene fusions, variable number tandem repeats, microsatellite repeats, trinucleotide repeat expansion data, splice variants and haplotypes associated with the complex disorder.

5. The method of claim 1, wherein the complex disorder is hypertension.

6. The method of claim 1, wherein the complex trait is blood pressure, including, but not limited to, systolic blood pressure, diastolic blood pressure, and pulse pressure.

7. A computer-based genomic annotation system comprising non-transitory memory configured to store instructions and at least one processor coupled with the memory, the processor configured to:
a. receive genome-wide variant data from a plurality of individuals with hypertension and a plurality of individuals without hypertension, or a plurality of individuals in which blood pressure is measured as a continuous variable, including, but not limited to, systolic blood pressure, diastolic blood pressure, and pulse pressure; and
b. identify genes related to sodium and potassium biology from a plurality of data sources, including but not limited to, biological pathways from ontological databases encompassing processes involved in the absorption, transport, action, or excretion of sodium and/or potassium, genes linked to sodium and potassium via evidence amassed in scientific literature, or genes for which expression or function is correlated with treatment by sodium, potassium, or a pharmacological agent that modulates either of these.

8. The computer-based genomic annotation system of claim 8, further comprising a separate computational process to interrogate the genotype of an individual subject to identify individuals sensitive to sodium and/or potassium interventions to lower blood pressure:
a. receiving genome-wide variant data from a biological sample from the subject;
b. calculating a predictive polygenic score for the clinically relevant gene-set related to sodium and/or potassium from the subject's genome wide variants data; and
c. selecting at least one intervention related to sodium and/or potassium, wherein a predictive polygenic score calculated that is elevated relative to a reference value indicates that the subject may be sensitive to the at least intervention including, but is not limited to, a pharmacological agent, lifestyle or dietary intervention, or non-prescription supplement; designed to achieve at least one of the following: lower sodium, raised potassium, or a reduction an individual's sodium-to-potassium ratio.
